(19) United States
(12) Patent Application Publication
(10) Pub. No.: US 2008/0147147 A1
(43) Pub. Date: Jun. 19, 2008

(54) VEIN LOCATING DEVICE FOR VASCULAR ACCESS PROCEDURES

(75) Inventors: David M. Griffiths, Pittsburgh, PA (US); Arthur E. Uber, Pittsburgh, PA (US); Page Cedarholm, Pittsburgh, PA (US); Adrienne Fazio, Bethel Park, PA (US); Michael J. Yanniello, Cheswick, PA (US)

Correspondence Address: GREGORY L BRADLEY;MEDRAD INC, ONE MEDRAD DRIVE, INDIANOLA, PA, 15051, US

(73) Assignee: MEDRAD, INC., Indianola, PA (US)

(21) Appl. No.: 11/612,147

(22) Filed: Dec. 18, 2006

(51) Int. Cl. A61N 5/06

(52) U.S. Cl. 607/88; 600/461

(57) ABSTRACT

A device for detecting a venous structure or vessels in a patient or animal. The device includes an optical source for transmitting optical energy into the tissue of the patient, an optical detector for detecting at least a portion of the optical energy that is transmitted into and reflected by the tissue, and an indicator operably associated with the optical source and the optical detector. The indicator is adapted to indicate relative changes in the detected reflection or scattering of the optical energy transmitted into the tissue of the patient.

### BACKGROUND OF THE INVENTION

This invention relates generally to vein locating or vein visualization devices, and more specifically to devices that use near-infrared (NIR) or ultrasound energy to locate or visualize venous structures in patients.

Intravenous (IV) access is problematic in many patients due to difficulty in finding and locating veins that are suitable. Many patients have veins that are not visible with the naked eye, or are beneath the surface of the skin so that they cannot be felt or seen. Patients with dark skin, and excess of subcutaneous fat, or with small or deep veins often fall into this category.

As reported by InfraRed Imaging Systems, Inc. of Columbus, Ohio, vascular access procedures rank as the most commonly performed, invasive, medical procedure in the U.S., with over 1.4 billion procedures performed annually (c 2005). These procedures also rank as the top patient complaint among clinical procedures. The overwhelming majority of vascular access procedures are performed without the aid of any visualization device and rely on what is observed through the patient's skin and by the clinician's ability to feel the vessel. Medical literature reports the following statistics: (1) a 28% first attempt IV failure rate in normal adults; (2) a 44% first attempt IV failure rate in pediatrics; (3) 43% of pediatric IVs require three or more insertion attempts; (4) a 23% to 28% incidence of extravasation/infiltration; (5) a 12% outright failure rate in cancer patients; and (6) 25% of hospital in-patients beyond three days experience difficult vascular access. See Brown P., “An I.V. Specialty team can mean savings for hospital and patient,” Journal of the National Intravenous Therapy Association, 17(5):387-388 (1984); Frey A M., “Success rates for peripheral IV insertion in a children's hospital,” Journal of Intravenous Nursing, 21(3):160-165 (1998); Palefski S. et al., “The infusion nurse and patient complication rates of peripheral short catheters: A prospective evaluation,” Journal of Intravenous Nursing, 24(2):113-123 (2001); Lininger R., “Pediatric peripheral IV insertion success rates,” Pediatric Nursing, 29(5):351-254 (2003); and Barton A. et al., “Improving patient outcomes through CQI: Vascular access planning,” Journal of Nursing Care Quality. 13(2):77-85 (1998), the contents of which are incorporated herein by reference.

A number of products for locating veins are known or currently available. These include products utilizing (1) ultrasound imaging, such as the Bard Site-Rite® 5 Ultrasound System marketed by Bard Access Systems, Inc. of Salt Lake City, Utah, (2) near-infrared (NIR) imaging, such as the IRIS Vascular Viewer marketed by InfraRed Imaging Systems, Inc. and the Vein Viewer Imaging System marketed by Luminetx Corporation of Memphis, Tenn., (3) liquid crystal thermal surface temperature measurement patches, such as the K-4000 Vena-Vue® Thermographic Vein Evaluator manufactured by Biosynergy, Inc. of Elk Grove Village, Ill., and (4) visible light illumination, such as the Venoscope® II Transilluminator/Vein Finder and the Neonatal Transilluminator marketed by Venoscope, L.L.C. of Lafayette, La., and the Veinlite®, Veinlite LED™, Veinlite EMS™ and Veinlite PEDI™ manufactured by TransLite, LLC of Sugar Land, Tex.

Others have performed experiments using optical light fibers that are moved over the skin to generate vessel maps based on spatially resolved reflectance at the skin surface. See Fridolin et al., “Optical Non-Invasive Techniques for Vessel Imaging: I. Experimental Results,” Phys. Med. Biol. 45, 3765-3778 (2000) and Fridolin et al., “Optical Non-Invasive Techniques for Vessel Imaging: II. A Simplified Photo Diffusion Analysis,” Phys. Med. Biol. 45, 3779-3792 (2000), the contents of which are incorporated herein by reference.

The currently-available vein-locating products often include imaging equipment and displays. In addition, imaging equipment, though portable, often must reside on a cart or stand, making transport difficult. Liquid crystal surface temperature patches work in some cases; however, they are difficult to use and may not work on patients that have poor limb circulation, small veins, or that have a greater than average subcutaneous fat layer near the IV access site.

The inadequacies of current vascular access practices significantly compromise patient care and contribute to rising healthcare costs. Multiple access attempts and outright failures delay patient treatment, frustrate healthcare professionals, and increase the likelihood of downstream complications and expense.

### SUMMARY OF THE INVENTION

The invention provides an improved vein-locating or vein-visualization device that is intended for use during intravenous access medical procedures. The vein-locating or vein-visualization device of the present invention is, in one aspect, a device that is used in a similar manner to a construction stud finder to identify regions under the skin where there is a change in light absorption or blood flow.

In another aspect, the invention utilizes near-infrared (NIR) energy or ultrasound energy to “see” various to various depths, such as several cm, within tissue. Veins contain de-oxygenated hemoglobin, which has a near infrared absorption peak at around 760 nm and a lesser, more broad absorption plateau over the range of 800 to 950 nm. There is a window of wavelengths in the near infrared region between 650 and 900 nm in which photons are able to penetrate tissue far enough to illuminate deeper structures beyond depths of 1 cm. In a preferred embodiment, the invention utilizes near-infrared wavelengths of approximately 880 to 890 nm for imaging subcutaneous veins in tissue.

In yet another aspect, the invention provides a self-contained, small, low-cost, and portable vein-locating or vein-visualization device for clinical use. In various embodiments, the present invention may include (1) a single infrared source and detector pair, (2) a single source and an array of detectors, or (3) an array of sources and an array of detectors.

In a further aspect, the present invention provides a device for detecting a surface or subsurface venous or vascular structure in a patient. The device includes an optical source for transmitting optical energy into the tissue of the patient, an optical detector for detecting at least a portion of the optical energy that is transmitted into and reflected and scattered by the tissue, and an indicator operably associated with the optical source and the optical detector. The indicator is adapted to indicate relative changes in the detected reflection of the optical energy transmitted into the tissue of the patient.

In still another aspect, the present invention provides a device for detecting a venous structure in a patient. The device includes an optical source for transmitting optical energy into the tissue of the patient, an optical detector for detecting at least a portion of the optical energy that is transmitted into and reflected by the tissue, and a removable coupler operably associated with the optical source and the optical detector.

In yet another aspect, the present invention provides a device for detecting a venous structure in a patient. The devices includes an optical source for transmitting optical energy into the tissue of the patient, an optical detector for detecting at least a portion of the optical energy that is transmitted into and reflected by the tissue, and indicator operably associated with the optical source and the optical detector. The indicator is adapted to indicate relative changes in the detected reflection of the optical energy transmitted into the tissue of the patient.

Further, the device includes a removable coupler operably associated with the optical source and the optical detector, one or more visible light illuminating sources adapted to be positioned near the skin surface of the patient to improve the ability to detect near-surface veins in the tissue, an amplifier operably associated with the optical detector, a modulator operably associated with the optical source and the amplifier, and a marking device to mark the location of the venous structure. The device may also provide for a guide for needle access.

By performing a simple reflectance measurement from the skin surface, it is possible to detect the location of a vein at depths of 4 mm or more below the skin surface. In a preferred aspect, the source and detector elements are incorporated within a hand held, battery operated, portable device that clinicians can use as a tool to help locate veins and thereby assist with IV access.

### BRIEF DESCRIPTION OF THE DRAWINGS

The present invention, and its presently preferred and alternate embodiments, will be better understood by way of reference to the detailed description herebelow and to the accompanying drawings, wherein:

FIG. 1 illustrates a first preferred embodiment of the present invention;

FIG. 2 illustrates a second preferred embodiment of the present invention;

FIG. 3a illustrates an alternate embodiment of the source and detector embodiment of FIGS. 1 and 2;

FIG. 3b illustrates another alternate embodiment of the source and detector embodiment of FIGS. 1 and 2;

FIG. 3c illustrates a histogram display for the source and detector embodiment of FIG. 3b;

FIG. 4 illustrates a third preferred embodiment of the present invention;

FIG. 5 illustrates a preferred embodiment of the coupler of the present invention;

FIG. 6 illustrates a fourth preferred embodiment of the present invention; and

FIG. 7 illustrates an electronics schematic for a fifth preferred embodiment of the present invention.

### DETAILED DESCRIPTION OF THE INVENTION

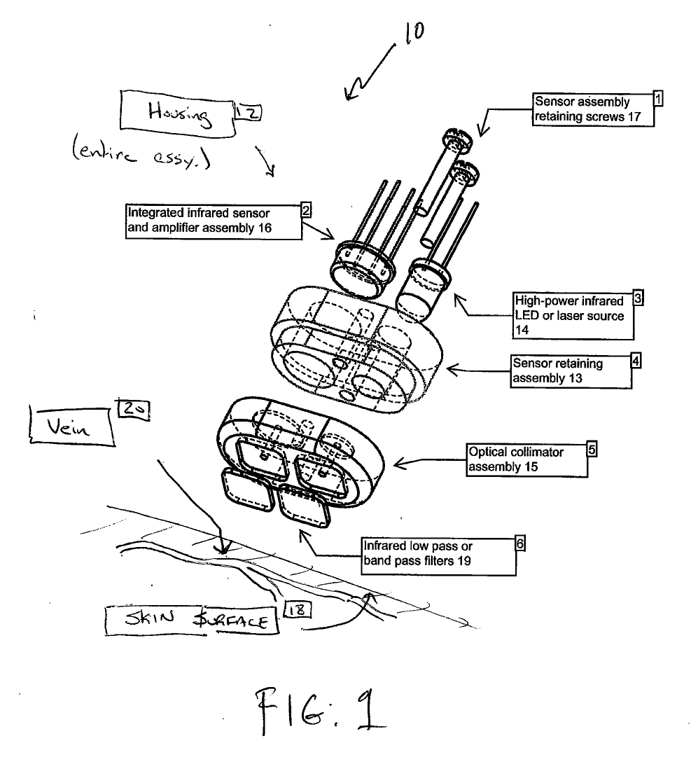

In one embodiment, as shown in FIG. 1, the vein-locating device 10 of the present invention preferably includes a housing 12 that contains an optical source 14 and an optical detector and amplifier assembly 16, which are adapted to be placed against or near the surface 18 of a patient's skin. In a preferred embodiment, the optical source 14 is a NIR source and the optical detector 16 is a NIR detector.

The housing 12 includes a sensor retaining assembly 13 and an optical collimator assembly 15 for retaining the optical source 14 and the optical detector 16. The housing 12 is held together with suitable fasteners, such as screws 17. The housing 12 further includes low-pass or band-pass filters 19, as explained further below.

The optical source 14 and the optical detector 16 are preferably oriented such that they are perpendicular to the surface 18 of the patient's skin and separated by several mm distance. When the optical source 14 (such as a NIR source) is activated, infrared photons travel through the patient's skin and tissue and are scattered and diffused. The detector 16 picks up some of this reflected and scattered light. When the source 14 is placed over a vein 20, the vein tissue absorbs some of the infrared light and the detector 16 picks up less reflected energy at those wavelengths. This causes a detectable signal change, which indicates the presence of the vein 20. Best results occur when the source 14 and detector 16 line up over the vein 20 (i.e., are in parallel with the direction of the vein 20), since this creates the longest absorption path. Variations in signal change due to a vessel or vein being located beneath the source-detector pair 14, 16 decrease logarithmically with distance. For a source and detector pair 14, 16, each having an aperture of about 1 mm, the optimal separation distance is approximately 5-6 mm. This will typically provide an imaging depth of at least 3-4 mm below the skin surface 18 of a patient.

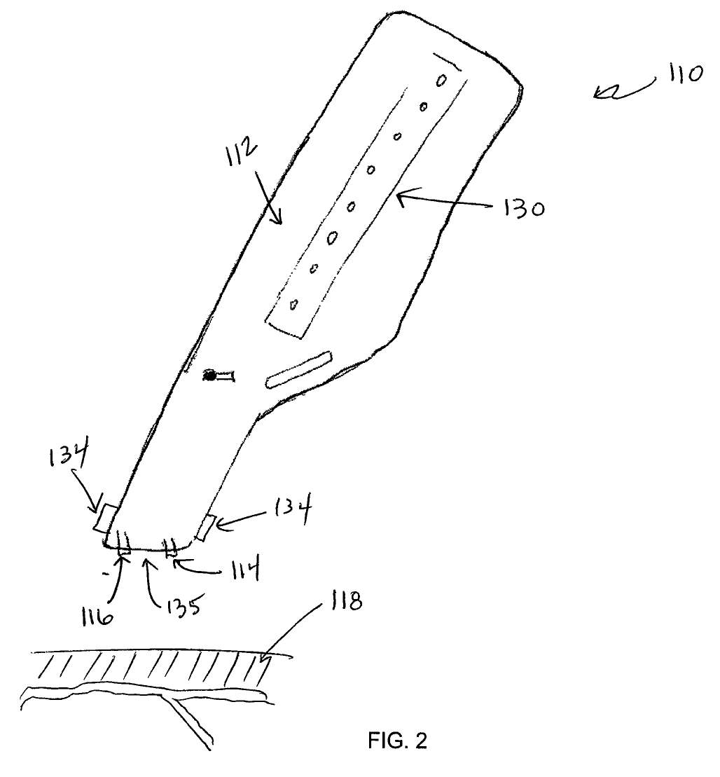

In another embodiment, as shown in FIG. 2, the device 110 of the present invention may also contain an indicator 130, such as an LED bar graph or other visual display, which indicates changes in reflection (absorption) from the source-detector 114, 116 arrangement. In operation, the clinician (operator) places the end of the device 110 on the patient and manually scans a region of the patient's skin to find a subsurface vein. When the probe is placed over a vein, the display shows changes in reflection (absorption) by (1) increasing or decreasing the length of the LED bar graph, (2) changing the intensity of the LED display, (3) changing the pulse period, and/or (4) some other visual indication. In addition to or instead of a visual display, the device 110 may have audible or tactile indicators.

Further, the housing 112 of the device 110 may also include one or more visible light illuminating sources 134, such as LEDs, which are disposed to be near the surface 118 of the skin, so that visual detection of near-surface veins is facilitated and/or improved. This also has the added benefit of serving as a power-on indication to the operator, since the NIR energy transmitted by the source 114 is not visible to the naked eye.

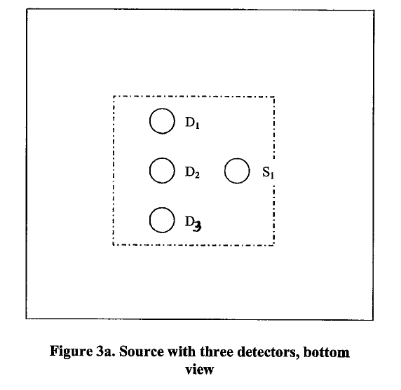

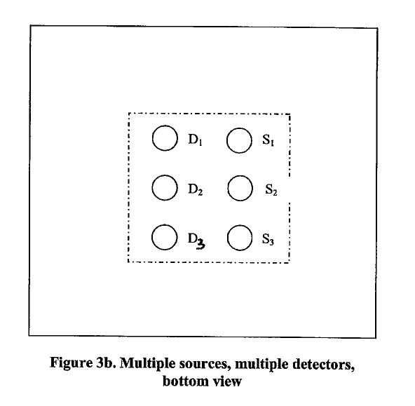

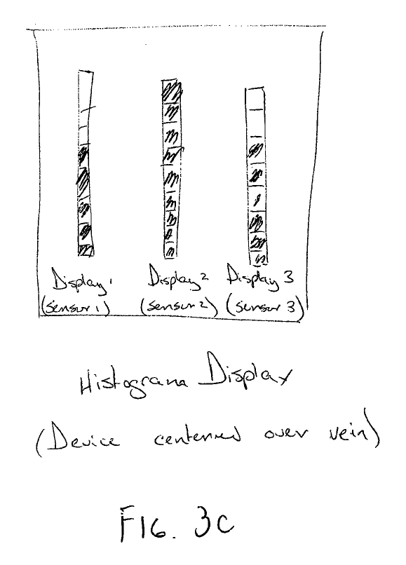

If, as shown in FIGS. 3a and 3b, the device 10, 110 utilizes multiple detectors D1-D3 and a single source S1 or multiple detectors D1-D3 and multiple sources S1-S3, it is possible to obtain additional information about vessel depth and orientation, as well as detect vessel movement during IV access.

In the embodiment shown in FIG. 3b, the sources S1-S3 can be turned on selectively or scanned, while the detector information is sent to a display. Information from multiple detectors D1-D3 (shown in FIGS. 3a and 3b) may be displayed on a histogram display (FIG. 3c) that shows changes in absorption (reflection) for each detector.

For example, as shown in FIG. 3a, the device 10, 110 of the present invention could use one source S1 and three detectors D1-D3 mounted in a linear fashion. This embodiment could be used to find and then indicate any movement or shifting of the vein during the IV access procedure/needle-stick. The device would be used to scan a region of the patient's skin until the middle indicator or bar graph associated with the middle detector D2 showed the most signal change. Then, with the vein finder device 10, 110 in place, the clinician may attempt to put in the IV device. If, during insertion, the vein shifts right or left, the adjacent bar graph indicators will show changes in signal intensity and possible vein movement. In another configuration, a single display may be used that shows condensed view of the measured signal intensity. For example, the product of signal change from two pairs of source-detectors may be displayed to give an indication of when both pairs are near a vessel, which gives some idea of vessel direction and alignment.

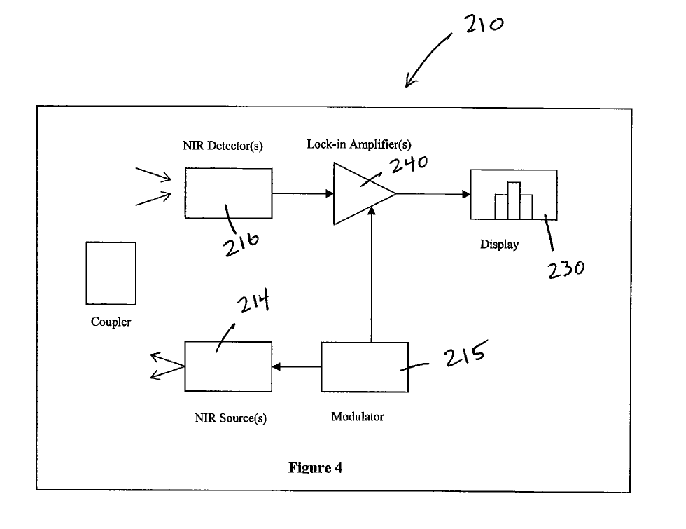

For increased sensitivity and depth, as shown in FIG. 4, the device 210 may use a lock-in amplifier 240 or synchronous detection scheme for the received optical signal. In this embodiment, the optical source(s) 214 is modulated or pulsed by the modulator 215, and the signals from the detector(s) 216 are gated or “locked-in” to the source frequency by the lock-in amplifier 240 when the reflected signal is measured. This approach minimizes noise effects from ambient light sources, such as indoor lighting or changing room lighting, because the device 210 only monitors the signal due to the device infrared source, not ambient sources. In addition, the optical detector(s) 216 may contain infrared low pass or band pass filters (as shown in FIG. 1), which block and reduce visible light interference and only let the infrared wavelengths of interest through to the detector(s) 216.

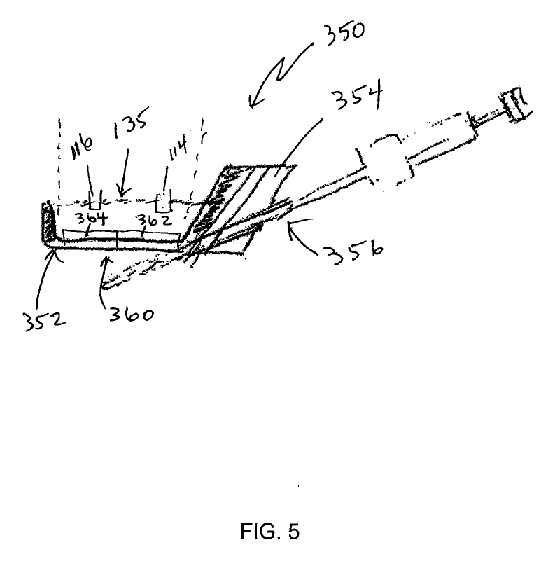

A key component of the vein finding device 10, 110, 210 is a per-patient sterile disposable coupler/needle guide 350, as shown in FIG. 5. The disposable coupler 350 fits over the patient contact end 135 (shown also in FIG. 2) of the device 10, 110, 210 and serves several purposes. First, the coupler 350 provides patient-to-patient isolation to minimize the spread of germs and other contamination between patients. Secondly, the coupler 350 provides a means to optically couple the source 14, 114, 214 and detector 16, 116, 216 to the skin surface and protect the optical source and detector elements from contamination and mechanical abrasion. The refractive index of the coupler 350 may be selected for the best match to the skin surface for transmitting and receiving light. Third, the coupler 350 may includes a means to collimate and steer the optical source and receive paths to optimize detection depth and resolution. Fourth, the coupler 350 may include optical shielding 352 against ambient infrared light sources, such as sunlight, incandescent lights, or fluorescent lights, which are commonly found in home and hospital environments where IV access procedures are typically performed. Fifth, the coupler 350 may include an integral needle guide 354 so that once the vein is located the operator may insert a catheter 356 or introducer needle (in the case of PICC lines) so that it is guided towards the vein identified by the device 10, 110, 210. The coupler 350 may contain multiple guides so that it can be used for a variety of needle entry angles, depending on the type of IV access to be performed. Sixth, the coupler 350 may also contain a means for holding a syringe and IV access device, as well as a spring loaded release mechanism, such as connected to a button, to release the access device into the tissue.

The coupler 350 or, alternatively, the source 14, 114, 214 and detector 16, 116, 216 may contain focusing elements or polarizing filters 360 to polarize the transmitted and received light. A linear polarizing filter 362 is placed on the source 14, 114, 214 and another linear polarizing filter 364 that is optically orthogonal to the first 362 is placed on the detector 16, 116, 216. The polarization of light is preserved in specular reflections and for certain geometries in single scattering events. Multiple Rayleigh-scattering events in tissue diffuse and depolarize the source. (Specular reflection is the perfect, mirror-like reflection of light (or sometimes other kinds of wave) from a surface, in which light from a single incoming direction is reflected into a single outgoing direction. Such behavior is described by the law of reflection, which states that the direction of outgoing reflected light and the direction of incoming light make the same angle with respect to the surface normal; this is commonly stated as θi=θr. This is in contrast to diffuse reflection, where incoming light is reflected in a broad range of directions.)

The polarizers 360 attenuate the light from specular reflections and near-field scattering events. The only light returning from the subject that can pass through the polarization filters 360 results from multiple scattering events occurring relatively deep (about 10 times the single scattering length) within the tissue. This scattered, depolarized light can be thought of as a virtual source that effectively back-illuminates any absorbing material in the foreground. This is linked to photon migration studies. The use of crossed polarizers gates photons by their migration path. Photons that have a longer diffusion path are more likely to pass through the filters and be received by the detector. This allows the device 10, 110, 210 to “see” more deeply into tissue. Also, the received signal will be more attenuated, so a more powerful source 14, 214, 216, such as a laser diode, may be used to obtain sufficient signal power/illumination.

To use the device 10, 110, 210, the operator first installs a disposable coupler 350. Next, they turn on the device, which activates the infrared source(s) 14, 114, 214, detector(s) 16, 116, 216 and any signal processing. Once the device has been activated, it is placed on the patient's skin, so that the disposable coupler 350 is in contact with the skin surface. An optical coupling gel may be used to obtain better optical signal match to the patient's tissue.

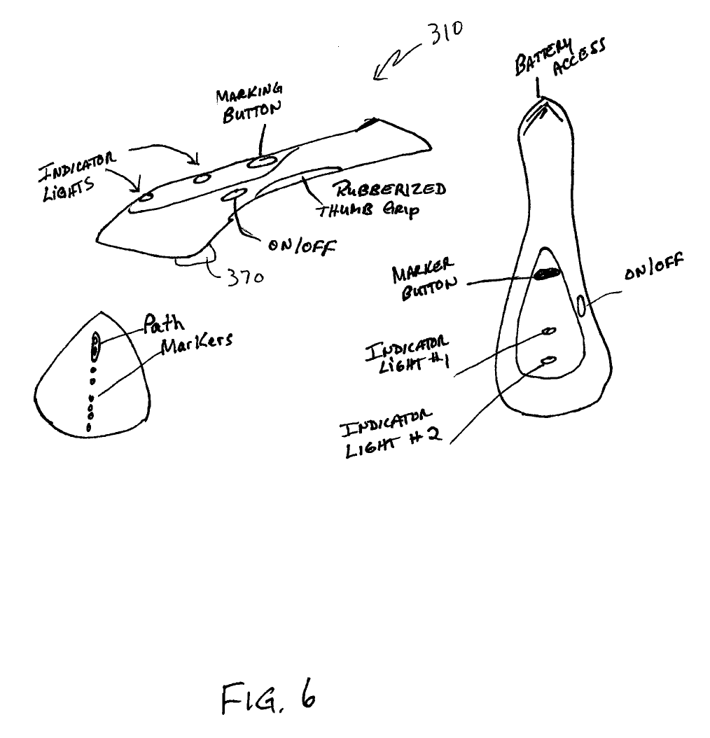

The device 10, 110, 210 may be placed on the patient's forearm or in the bend of the elbow (antecubital fascia), which are common places on the body for peripheral IV access, or the device is placed on another part of the patient's body. Next, the operator scans the device across the skin in the direction perpendicular to the expected longitudinal direction of the veins. By monitoring the indicator or display 130 for peaks in absorption, the operator locates a target region for IV insertion. The peaks of the indicator/display 130 represent the depth and size of the vessel below the source-detector arrangement. Next the operator rotates the device to establish vein direction. For a single source-detector configuration, the greatest absorption occurs when the source and detector are oriented in a line that is parallel and over the vein below. For a single source-multiple detector configuration, the vein direction is determined by the widest spatial spread of absorption, which indicates that the detectors are oriented in a line that is parallel and over the vein below. Once the vein location and direction are established, the operator may either mark the IV access site with a marking device or pen 370 (which may be provided by or incorporated in the device, as shown in FIG. 6), or use the needle guide to insert the catheter needle. The marking means may also consist of a marker material or object, such as a polymer label or a plastic part, which is deposited on the surface of the skin to act as landmarks for locating vessels detected with the device. Once the vein has been located and/or the IV needle placed, the device 10, 110, 210 may be turned off and the disposable coupler 350 discarded.

The device may also be connected to an image display such that the information from the device is presented on the display to present a map as the device is scanned over a region of interest within the tissue. For accurate display, the position and or scanning rate of the device must be captured in at least two dimensions.

The device may also project a line of visible light onto the surface of the skin over the region where the detected vessel is located, and aligned with the longitudinal direction of the vessel to identify vessel location.

For ease of use, the device 10, 110, 210 is preferably hand held, and may be designed so that during use it rests across the surface of the arm or area to be scanned. This way, the device must only be held down against the surface of the skin and is supported by the surface of the patient when the operator inserts the vascular access device.

As shown in FIG. 6, in an alternate embodiment the device 310 may also include a marking implement, such as an ink pen or stamping device 370 (as also shown in FIG. 2) so that the skin surface over the region where a vessel is detected can be marked for future reference. Marking can include registration marks, needle guidance marks, or other marks on or around the located vein. Ink marks can be temporary or made with permanent indelible ink. Marks may even be colorful and decorative for pediatric use. The marking material may also be composed of antiseptic, antibacterial, or anesthetic compounds. Making a physical impression in the skin may also perform marking. This way, the operator can put down the vein finding device 10, 110, 210 and access the vein according to standard practice, or the path of the vessel may simply be mapped for future reference.

In another embodiment, the device projects a line of visible light onto the surface of the skin, in the longitudinal direction of the vessel, when the vessel is detected.

In another embodiment, the device may use a an optical Doppler technique where the optical reflection is detected with a sensor, then signal processing is used to identify the portion of the reflection that is due to moving blood cells within the vessel. A similar technique is sometimes used for tissue perfusion measurements, however, it may also be possible to use measurement changes based on Doppler velocimetry to determine the location of larger near-surface vessels based on the relative measurements of blood cell flux as the cells move through tissue. Flux measured in larger veins will be greater than in surrounding tissue capillary flow. For this approach, a single coherent frequency stable optical source such as a laser is used. The optical source is then directed into the tissue, and the reflected signal is monitored with two or more sensors, where the interference between the two is used to provide a Doppler beat frequency which is then used to determine the velocity of particles moving beneath the surface. The beat frequency can be measured by a standard laser Doppler signal processor, a commercial digital photon correlator, or a fast digital correlator. See Tong P. et al., “Two-fiber-optic method of laser Doppler velocimetry,” NASA Tech Briefs (May 2002), the contents of which are incorporated herein by reference.

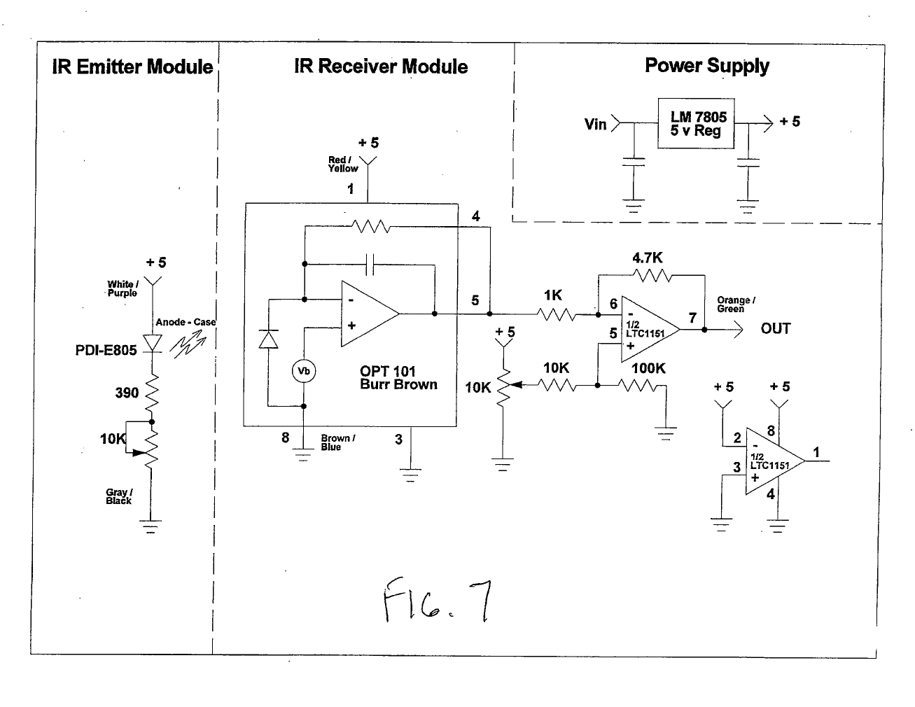

Electronic schematics for an IR emitter, an IR receiver and a Power Supply for another preferred embodiment of the invention are shown in FIG. 7.

Although the present invention has been described in detail in connection with the above embodiments and/or examples, it is to be understood that such detail is solely for that purpose and that variations can be made by those skilled in the art without departing from the invention. The components and features of the various embodiments of the invention can be assorted or combined as appropriate for the application. The scope of the invention is indicated by the following claims rather than by the foregoing description. All changes and variations which come within the meaning and range of equivalency of the claims are to be embraced within their scope.

What is claimed is:

1. A device for detecting a venous structure in a patient, comprising:
an optical source for transmitting optical energy into tissue;
an optical detector for detecting at least a portion of the optical energy that is transmitted into and reflected by the tissue; and
an indicator operably associated with the optical source and the optical detector, the indicator adapted to indicate relative changes in the detected reflection and scattering of the optical energy transmitted into the tissue of the patient.

2. The device of claim 1 wherein the indicator comprises an LED display.

3. The device of claim 2 wherein the LED display is an LED bar graph.

4. The device of claim 3 wherein the length of the LED bar graph increases or decreases in proportion to the relative changes in the detected reflection of the optical energy.

5. The device of claim 2 wherein the intensity of the LED display changes in proportion to the relative changes in the detected reflection of the optical energy.

6. The device of claim 1, further comprising one or more visible light illuminating sources adapted to be positioned near the skin surface of the patient to improve the ability to detect near-surface veins in the tissue.

7. The device of claim 6 wherein the one or more light illuminating sources are light-emitting diodes or are frequency or temporally modulated.

8. The device of claim 1 wherein the optical source and the optical detector are separated by approximately 5-6 mm.

9. The device of claim 1 wherein the indicator provides visual, audible, or tactile information to the operator.

10. The device of claim 1, further comprising a marking device to mark the location of the venous structure.

11. The device of claim 10 wherein the marking device is an ink marking device.

12. The device of claim 1, further comprising an amplifier operably associated with the optical detector and a modulator operably associated with the optical source and the amplifier.

13. The device of claim 12 wherein the amplifier is a lock-in amplifier or uses asynchronous or synchronous detection of the optical signal modulation.

14. The device of claim 1 wherein the optical detector comprises a plurality of optical detectors.

15. The device of claim 1 wherein the optical detector comprises a plurality of optical detectors and the optical source comprises a plurality of optical sources.

16. A device for detecting a venous structure in a patient, comprising:
an optical source for transmitting optical energy into the tissue of the patient;
an optical detector for detecting at least a portion of the optical energy that is transmitted into and reflected by the tissue; and
a removable coupler operably associated with the optical source and the optical detector.

17. The device of claim 16 wherein the coupler comprises a needle guide adapted to receive a catheter or introducer needle for insertion into the venous structure.

18. The device of claim 16 wherein the coupler comprises one or more focusing elements or polarizing filters.

19. The device of claim wherein the coupler is adapted to optically couple the optical source and the optical detector to the skin of the patient.

20. A device for detecting a venous structure in a patient, comprising:
an optical source for transmitting optical energy into the tissue of the patient;
an optical detector for detecting at least a portion of the optical energy that is transmitted into and reflected by the tissue;
an indicator operably associated with the optical source and the optical detector, the indicator adapted to indicate relative changes in the intensity of the detected reflection of the optical energy transmitted into the tissue of the patient;
an indicator operably associated with the optical source and the optical detector, the indicator adapted to indicate relative changes in the frequency of the detected reflection of the optical energy transmitted into the tissue of the patient;
a removable coupler operably associated with the optical source and the optical detector;
one or more visible light illuminating sources adapted to be positioned near the skin surface of the patient to improve the ability to detect near-surface veins in the tissue;
an amplifier operably associated with the optical detector;
a modulator operably associated with the optical source and the amplifier;
a marking device to mark the location of the venous structure; and
a guiding device to guide the location of an vascular access device to the venous structure.
